(19) United States
(12) Patent Application Publication
(10) Pub. No.: US 2021/0022668 A1
(43) Pub. Date: Jan. 28, 2021

(54) FEEDBACK SYSTEMS AND METHODS FOR GAIT TRAINING FOR PEDIATRIC SUBJECTS AFFLICTED WITH GAIT DISORDERS

(71) Applicants: Kessler Foundation Inc., East Hanover, NJ (US); Children's Specialized Hospital, New Brunswick, NJ (US)

(72) Inventors: Peter John Barrance, Stirling, NJ (US); Nuno Miguel Moreira Cancela Oliveira, Hattiesburg, MS (US); Naphtaly Ehrenberg, Teaneck, NJ (US); JenFu Cheng, Watchung, NJ (US); Sheila Blochlinger, Green Brook, NJ (US)

(21) Appl. No.: 16/937,666

(22) Filed: Jul. 24, 2020

Related U.S. Application Data
(60) Provisional application No. 62/878,033, filed on Jul. 24, 2019.

(51) Int. Cl. A61B 5/00; A61B 5/11; A63B 22/02; A63B 24/00

(52) U.S. Cl. CPC A61B 5/486; A61B 5/112; A61B 5/1121; A61B 5/7405; A61B 5/742; A61B 2562/0219; A63B 22/02; A61B 5/6828; A61B 5/6829; A63B 24/0062; A61B 5/7455

(57) ABSTRACT

Provided herein is a system or method for biofeedback for gait training in a subject in need thereof, involving sensors capable of being put on the thigh, shank and heel of the subject, and two separate output displays shows knee flexion data and knee extension data in real-time.

### CROSS-REFERENCE TO RELATED APPLICATION

This application claims the benefit of U.S. Provisional Patent Application No. 62/878,033, filed Jul. 24, 2019, the contents of which are hereby incorporated herein in its entirety.

### FIELD OF THE INVENTION

The invention relates to systems and methods for use in improving gait in subjects afflicted with gait disorders.

All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

The 2012 Americans with Disabilities Report estimated that around 580,000 children in the United States under the age of 15 experience difficulty with walking or running¹. Such disability impacts functional mobility and can affect social development and engagement. Pathological gait patterns are also associated with long term development of musculoskeletal injuries such as osteoarthritis³⁻⁴ and these factors are particularly critical when onset is at birth or during childhood. Cerebral palsy (CP)⁵ and, with a much lower incidence, spinal cord injury (SCI)⁶⁻⁷ are two diagnoses that are frequently associated with such disability.

Altered knee motion is one of the most common gait deviations in pediatric populations afflicted with gait disorders. The potential for pediatric gait retraining using visual feedback based on knee kinematic patterns has not been sufficiently studied.

Altered sagittal knee motion is one of the most common deviations associated with reduced gait function⁸⁻⁹. Nieuwenhuys¹⁰ and Krawetz and Nance¹¹ reported a variety of temporal and spatial deviations in knee flexion patterns associated with CP and SCI. These deviations from normative gait have severe consequences for gait function. For example, reduced knee extension in terminal swing leads to shortened stride length which results in a decrease in velocity, whereas a reduced peak knee flexion during mid swing is associated with loss of foot clearance and can increase the risk of falls^{8,12}.

Both surgical and therapeutic approaches have been developed to address gait deviations. Most therapeutic approaches have employed repetitive training provided by treadmill walking, complemented by either manual or robotic mechanical guidance of limb segments towards normalized temporal and spatial gait parameters. Using such methods, reduced deviations in knee kinematics and improvements in functional measures have been demonstrated in ambulatory CP¹³ and SCI¹⁴ patients. These methods are inherently resource intensive; for instance, both robotic training equipment and commitments of manual therapeutic training time are expensive. Biofeedback retraining paradigms are based on the patient's active interpretation and response to feedback cues of gait pattern deviations. They require sensing, processing, and output (e.g. visual, haptic, or auditory) technologies that tend to be less expensive than the hardware required to generate and control assistive forces. Whereas active limb assistance will continue to be a necessity in some patients, there is evidence that within both SCI and CP populations there exist subsets of patients that exhibit pathological kinematics despite normal, or near normal, voluntary joint ranges of motion. In the CP population, a recent study¹⁶ showed that a pattern of increased knee flexion at initial contact was significantly correlated with a deficit in selective motor control for the lower extremity. In contrast with other patterns that were correlated with structural findings such as muscle contractures, this learned dysfunctional motor pattern may be addressable by feedback-based gait retraining.

During biofeedback-based gait training, real-time visual¹⁷, auditory¹⁸, or haptic¹⁹ information augments native sensory and visual feedback. Successful use of visual feedback relies on the sensory integration of visual signals to modify specific motor behaviors²⁰, and several studies have reported that adults and pediatrics differ in the speed and manner of processing and integrating visual, vestibular, and somatosensory information²¹⁻²³. Moreover, successfully responding to feedback cues depends on functional task complexity²⁴, which gauges the level of difficulty of the task in reference to the cognitive capacities of the user. On the one hand, while varying success has been reported in the use of biofeedback modalities that focus on one specific target parameter resulting from the gait cycle²⁵⁻²⁷, these techniques do not target deviations in other parts of the cycle, and might induce new deviations to the learned pattern. On the other hand, pattern based training with whole gait cycle feedback offers the advantage of specificity of guidance toward a desired target pattern, but the task of appropriately responding represents higher functional task complexity.

While other groups have studied the response to visual feedback based on the entire gait cycle in adults^{15, 17}, the inventors have uniquely advanced the field in the study and development of such whole gait cycle feedback in pediatrics. In a study published by the inventors²⁸, three sensors were fixed to the dominant lower limb of twelve typically developing children and adolescent participants. One sensor was placed on the anterior thigh, a second sensor was placed on the posterior shank, and a third sensor was attached to the heel of the participant's shoe. Participants were asked to view a single feedback needle display which moved in proportion to the sum of squared angle error across the cycle relative to a novel target pattern, and adopt gait modifications in response to the display. This study showed that maximum knee flexion during typically developing pediatric gait was directed toward specific targets in response to the display; however, overall cycle error was not reduced. The finding that the measure of overall convergence to the target (i.e. overall cycle error) was not improved demonstrates that innovation in this field is required.

### SUMMARY OF THE INVENTION

The present invention is directed to a system for biofeedback for gait training of a subject in need thereof, comprising sensors for measuring relative positions of a subject's thigh, shank and heel of at least one leg, a processing unit and two separate indicators, such as, for example, output displays. Such system being configured to measure knee flexion and knee extension and generating corresponding knee flexion and knee extension data signals for processing by the processing unit to generate and transmit corresponding knee flexion and knee extension indicator signals to respective ones of a first indicator, e.g., output display, indicating the knee flexion and a second indicator, e.g., output display, indicating knee extension in substantially real time. In one embodiment, such indicator signals represent the measured knee flexion data and knee extension data relative to predetermined reference knee flexion and extension data.

The invention is further directed to a method of biofeedback for gait training of a subject in need thereof, comprising the steps of (a) applying sensors to the thigh, shank and heel on at least one leg of the subject, wherein such sensors are configured to measure data for determining knee flexion and knee extension data; (b) processing by a processing unit knee flexion and knee extension data, and transmitting biofeedback indicator signals to two separate output indicators such as, for example, displays, wherein the first display depicts the measured knee flexion and the second display depicts the measure knee extension; (c) instructing the subject to adapt gait in real time based on feedback data. In another embodiment, such indicators signals represent the measured knee flexion data and knee extension data relative to predetermined reference knee flexion and extension data.

### BRIEF DESCRIPTION OF THE DRAWINGS

The drawings described below are for illustrative purposes only and are not intended to limit the scope of the invention.

FIGS. 1a and 1b depict gait exemplary retraining inertial sensor setup and feedback interface.

FIGS. 2a and 2b depict a representative learning effect of the participant to the feedback gait retraining.

FIG. 3 depicts an exemplary flow diagram beginning with the measurement of knee angle data and ending with the display feedback and scoring.

FIG. 4 depicts a representative flow diagram ending in the knee angle data.

FIG. 5 depicts exemplary information concerning Dynamic Time Warping and the extension and flexion phases.

FIG. 6 depicts representative information for converting error measures to a display.

FIG. 7a depicts an overview of the representative drawings of FIGS. 7b-7f FIG. 7b illustrates the gait pattern and feedback associated with an exemplary increased flexion at initial contact. FIG. 7c illustrates the gait pattern and feedback associated with an exemplary decrease in knee movement. FIG. 7d illustrates the gait pattern and feedback associated with a representative low knee flexion in flexion phase (stiff knee gait). FIG. 7e illustrates the gait pattern and feedback associated with a representative excessive flexion in extension phase (crouch gait). FIG. 7f illustrates a representative gait pattern and feedback associated with normative gait pattern.

### DETAILED DESCRIPTION

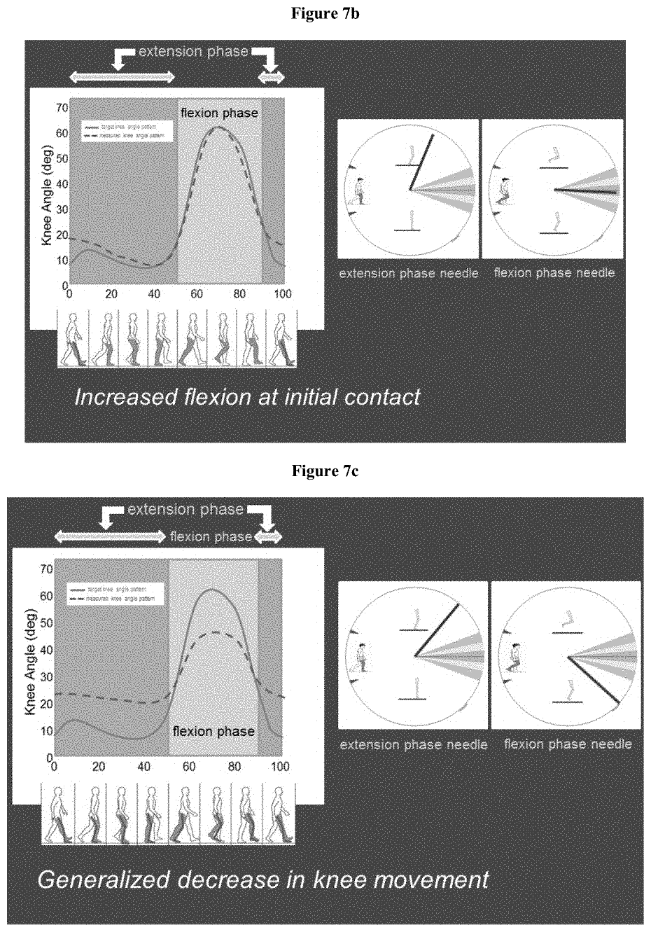

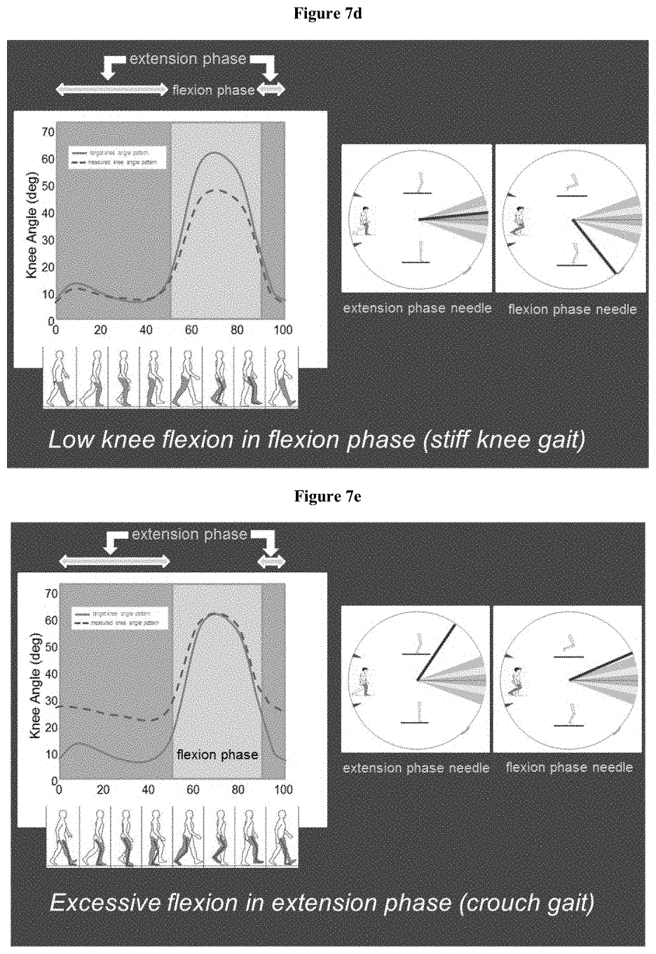

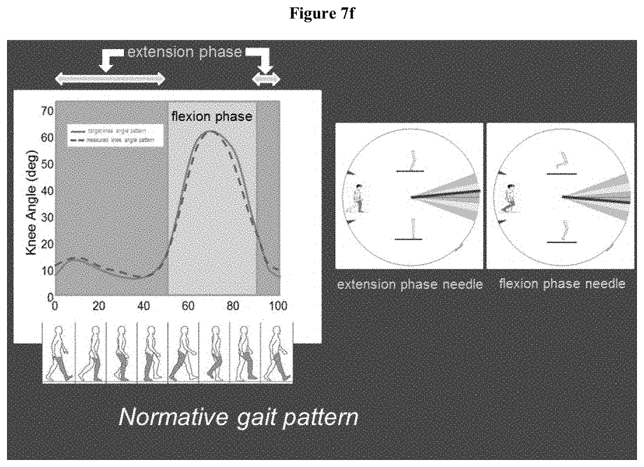

The invention is based in part on the discovery that by using a real-time pattern-based kinematic feedback gait retraining platform and displaying the feedback in a particularly advantageous manner, pediatric patients with gait disorders can improve their gait patterns. For example, FIGS. 7b to 7e depict typical gait patterns of a pediatric subject with a gait disorder, and feedback that can be provided to the subject in response to these gait patterns in order to improve the subject's gait pattern.

### Definitions

The articles “a” and “an” are used in this disclosure to refer to one or more than one (i.e., to at least one) of the grammatical object of the article.

A “subject” is a human, and the terms “subject” and “patient” are used interchangeably herein.

The term “treating,” with regard to a subject, refers to improving at least one symptom of the subject's disorder. Treating can be curing, improving, or at least partially ameliorating the disorder. For example, improving a subject's gait pattern would be considered treating the subject.

As used herein, “real time” or “real-time” generally refers to data that is presented as it is required. In this application, data presented in real-time is generally presented to the patient within seconds after the data is obtained and processed. For example, data presented in real-time may be presented after about 0 seconds, 0.25 seconds, 0.50 seconds, 1.0 second, between 0 and 5 seconds, between 0 and 10 seconds, or between 0 and 15 seconds after it is obtained and processed. Data presented in real-time need not be kept or stored, although keeping or storing may occur simultaneously to presenting the data in real-time.

In one embodiment of the invention, a system is provided for biofeedback for gait training in a subject in need thereof, comprising sensors attachable to the thigh, shank and heel of the subject, a processing unit and two separate feedback indicators, such as output displays. Such system being configured to measure knee flexion and knee extension and generating corresponding knee flexion and knee extension data signals for processing by the processing unit to generate and transmit corresponding knee flexion and knee extension indicator signals to respective ones of a first indicator, e.g., output display, indicating the knee flexion and a second indicator, e.g., output display, indicating knee extension in substantially real time. In one embodiment, such indicators signals represent the measured knee flexion data and knee extension data relative to predetermined reference knee flexion and extension data.

In another embodiment of the invention, a system is provided for biofeedback of gait training of a subject in need thereof, comprising: (a) sensors configured to be releasably attachable to at least one leg of the subject, said sensors configured to provide leg positional information during at least motion of the at least one leg during a walking motion of the at least one leg; (b) a processing unit for receiving the positional information from the sensors and for generating data indicative of knee flexion angles and knee extension angles during at least the walking motion of the at least one leg; and for generating corresponding respective knee flexion and knee extension angles training signals based on the generated data; and (c) at least one feedback unit configured to receive the generated training signals to provide feedback to the subject during at least the walking motion of the at least one leg, the feedback including at least a first component indicative of the subject's knee flexion angles during at least the walking motion of the at least one leg, and a second component indicative of the subject's knee extension angles during at least the walking motion of the at least one leg.

In another embodiment of the invention, provided is a system for biofeedback of gait data, wherein the processing unit is further configured to generate the training signals indicative of knee flexion angles relative to corresponding predetermined knee flexion reference angles.

In another embodiment of the invention, provided is a system for biofeedback of gait data, wherein the processing unit is further configured to generate the training signals indicative of knee extension angles relative to corresponding predetermined knee extension reference angles.

In another embodiment of the invention, provided is a system for biofeedback of gait data, wherein the at least one feedback unit is at least one of a display, projector, sound generator or haptic feedback device.

In another embodiment of the invention, provided is a system for biofeedback of gait data, wherein said sensors comprise at least one first, second and third sensors configured to be releasably attachable to respective one's thigh, shank and heel of the at least one leg of the subject.

In another embodiment of the invention, provided is a system for biofeedback of gait data, further comprising a treadmill positioned in proximity to the feedback device.

In another embodiment of the invention, provided is a system for biofeedback of gait data, wherein said sensors comprise at least one of inertial, magnetic, electromagnetic, micro-electromechanical or optical circuits configured to sense leg position.

In another embodiment of the invention, provided is a method of biofeedback for gait training of a subject in need thereof, comprising the steps of: (a) releasably attaching sensors to at least one leg of the subject, said sensors being configured to provide leg positional information during at least motion of the at least one leg during a walking motion of the at least one leg; (b) transmitting by the sensors to a processing unit, leg positional information during at least motion of the at least one leg during a walking motion of the at least one leg; (c) generating by the processing unit based on the leg positional information, data indicative of knee flexion angles and knee extension angles during at least the walking motion of the at least one leg; (d) generating by the processing unit, respective knee flexion and knee extension training signals based on the generated data; (e) at least one feedback unit receiving by at least one feedback unit, the generated training signals; and (f) providing feedback by the at least one feedback unit during at least the walking motion of the at least one leg based on the training signals, the feedback including at least a first component indicative of the subject's knee flexion angles during at least the walking motion of the at least one leg, and a second component indicative of the subject's knee extension angles during at least the walking motion of the at least one leg.

In another embodiment of the invention, provided is a system for biofeedback of gait data, wherein the step of generating the training signals generates training signals indicative of knee flexion angles relative to corresponding predetermined knee flexion reference angles.

In another embodiment of the invention, provided is a system for biofeedback of gait data, wherein the step of generating the training signals generates training signals indicative of knee extension angles relative to corresponding predetermined knee extension reference angles.

In another embodiment of the invention, provided is a system for biofeedback of gait data, wherein the feedback generating step generates feedback of at least one of a display, projector, sound generator or haptic feedback.

In another embodiment of the invention, provided is a system for biofeedback of gait data which further comprises the subject walking on a treadmill positioned in proximity to a device providing the feedback.

In another embodiment of the invention, provided is a system for biofeedback of gait data, wherein the step of releasably attaching sensors to at least one leg of the subject further comprises releasably attaching the sensors to respective one's thigh, shank and heel of the at least one leg of the subject.

In an additional embodiment of the invention, a method is provided of biofeedback of gait training of a subject in need thereof, comprising the steps of (a) applying sensors to the thigh, shank and heel on at least one leg of the subject, wherein such sensors are configured to measure data for determining knee flexion and knee extension data; (b) processing by a processing unit knee flexion and knee extension data, and transmitting biofeedback indicator signals to two separate output indicators such as, for example, displays, wherein the first display depicts the measured knee flexion and the second display depicts the measured knee extension; (c) instructing the subject to adapt gait in real time based on feedback data. In another embodiment, such indicators signals represent the measured knee flexion data and knee extension data relative to predetermined reference knee flexion and extension data.

In another embodiment of the invention, provided is a system or method of biofeedback of gait data, wherein the subject is afflicted with a gait disorder, cerebral palsy (CP), or hemiplegic CP.

In another embodiment of the invention, provided is a system or method of biofeedback of gait data, in which the subject is afflicted with CP and/or persistent gait deviations but has previously undergone successfully clinical management of spasticity and contractures.

In another embodiment of the invention, provided is a system or method of biofeedback of gait data, in which the subject is afflicted with injuries to the central nervous system such as incomplete spinal cord injury or stroke or the subject is an aged individual, such as an individual greater than 70 years old or greater than 75 years old.

In another embodiment of the invention, provided is a system or method of biofeedback of gait data, wherein the subject is less than 18 years old, less than 16 years old, less than 14 years old less than 12 years old, or greater than 7 years old and less than 16 years old.

In another embodiment of the invention, provided is a system or method of biofeedback of gait data, wherein the subject's gait pattern has (a) increased flexion at initial contact of the gait cycle compared to a normative gait pattern; (b) a generalized decrease in knee movement compared to a normative gait pattern; (c) a low knee flexion in the flexion phase of the gait cycle compared to a normative gait pattern; (d) an excessive flexion in extension phase of the gait cycle compared to a normative gait pattern; or (e) an insufficient knee flexion in the mid-swing phase cycle compared to a normative gait pattern.

In another embodiment of the invention, provided is a system or method of biofeedback of gait data, wherein the sensors are applied or are capable of being applied to the subject's dominant limb, optionally determined by the Waterloo Footedness Questionnaire.

In another embodiment of the invention, provided is a system or method of biofeedback of gait data, wherein the sensors are inertial sensors.

In another embodiment of the invention, provided is a system or method of biofeedback of gait data, wherein the sensors measure or are capable of measuring three-dimensional segment accelerations during gait.

In another embodiment of the invention, provided is a system or method of biofeedback of gait data, wherein the thigh sensor is located or to be located at the midpoint of the line from the patella to the anterior superior iliac spine and the thigh sensor's long axis was perpendicular to the line.

In another embodiment of the invention, provided is a system or method of biofeedback of gait data, wherein the shank sensor is located or to be located on the most prominent section of the gastrocnemius parallel to the thigh.

In another embodiment of the invention, provided is a system or method of biofeedback of gait data, wherein the knee extension data comprises data concerning the subject's late swing, early, and mid-stance phases of a gait cycle.

In another embodiment of the invention, provided is a system or method of biofeedback of gait data, wherein the knee flexion data comprises data concerning the subject's terminal stance, early and mid-swing phases of a gait cycle.

In another embodiment of the invention, provided is a system or method of biofeedback of gait data, wherein the first output display comprises a flexion error feedback needle.

In another embodiment of the invention, provided is a system or method of biofeedback of gait data, wherein the second output display comprises an extension error feedback needle.

In another embodiment of the invention, provided is a system or method of biofeedback of gait data, wherein the biofeedback is less sensitive to small variations in timing.

In another embodiment of the invention, provided is a method of biofeedback of gait data, further comprising instructing the subject to walk.

In another embodiment of the invention, provided is a method of biofeedback of gait data, wherein the method is performed periodically.

In another embodiment of the invention, provided is a method of biofeedback of gait data, wherein the method is performed periodically at least once per week, at least once every two weeks, at least once a month, at least once every 3 months, at least once every 6 months, at least once every year.

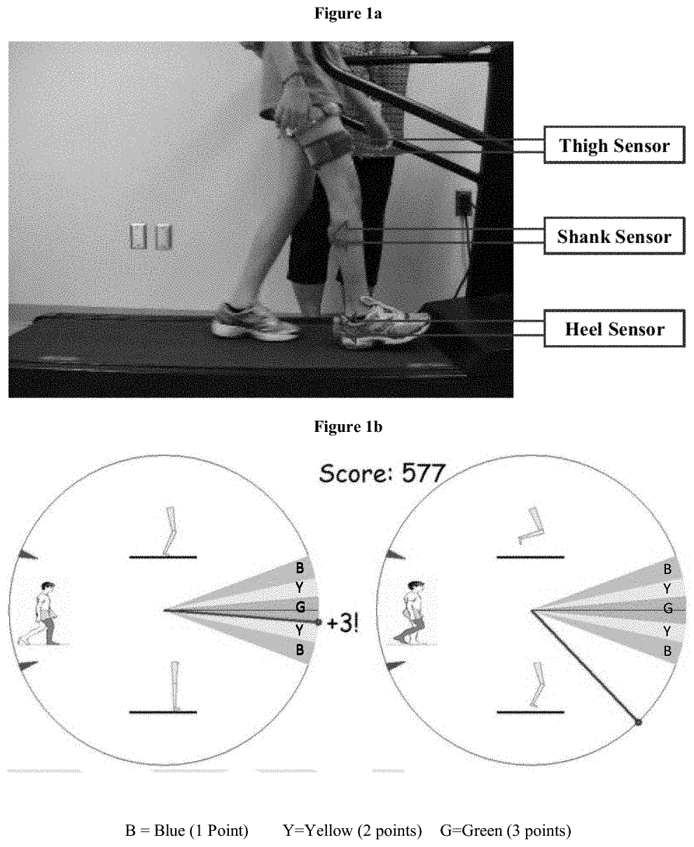

In another embodiment, three sensors may be used, with one sensor placed on the anterior aspect of the thigh, and a second sensor placed on the posterior aspect of the shank and a third sensor attached to the heel of the shoe. Knee flexion angles may be determined from the data provided by such sensors by a processing unit and then displayed on two displays in accordance with this disclosure. Such displays may be updated in real-time, for example, after approximately 25% of the gait period had elapsed to reflect the previous stride, optionally including, for example, different types of information as shown in FIG. 1b.

In a further embodiment, an exemplary platform to isolate the feedback according to extension and flexion roles in the gait cycle and this platform was evaluated on a pediatric patient with cerebral palsy. Three inertial sensors were placed on the participant's paretic lower limb. Based on real-time processing by a processing unit of data collected from the sensors, the exemplary platform provided concurrent kinematic feedback on separate indicators depicted on a computer monitor.

Representative dynamic time warping was used to generate two warped signals to attenuate temporal offsets between the measured and the target knee flexion patterns. On the feedback interface, the left panel showed the participant's extension period that includes the late swing, early and mid-stance phases of a gait cycle; the right panel showed the participant's flexion period that includes the terminal stance, early and mid-swing phases of a gait cycle. The knee flexion pattern for the most recent stride was compared with a reference target pattern.

During such evaluation, the participant presented stiff knee gait pattern on his paretic side (right), with insufficient knee flexion in the mid-swing phase. After calibration, the participant walked on the treadmill for 1 minute while his knee flexion pattern in natural walking was recorded as the baseline data. The participant then experienced a single session of gait retraining which included repeated (three) training bouts of 3-minute Feedback (FB) and 3-minute Non-Feedback (NFB) trials. The root-mean-square error (RMSE) was calculated and the mean knee flexion angle curve was compared with the target knee flexion angle curve.

Feedback was provided in two phases of the cycle including rewarding the participant for appropriate leg bending and straightening. Additional processing was also employed to generate the feedback signals to make them more forgiving of small timing variations and more reflective of the overall pattern similarity.

Reduction in knee flexion angle RMSE relative to baseline was observed in all trials with and without feedback. Adaptation to extension and flexion period error-based feedback showed increased swing phase knee flexion and maintenance of appropriate extension through majority of stance. Thus, the participant's knee pattern was prominently improved in the first feedback trial and retained without feedback, which indicated that the real-time visual feedback gait retraining is useful in correcting and refining specific motor patterns in terms of individualized training.

In a further embodiment, a processing unit of the system processes signals from inertial sensors (IMU) placed on one limb and to generate indicator signals for providing to an indicator for providing feedback. Such indicators may be in the form of a display depicting two feedback needles or other level indicators and scoring functionality. This representative feedback method measures one anatomical angle, specifically, knee flexion angle in real time. The knee angle measured by the IMUs is defined as the difference between the thigh and shank sensors' ‘Roll’ rotation. It should be understood that the use of providing such feedback in the form of displays is for illustration purposes only and that other forms of feedback based on the indicator signals are likewise suitable, including audio or haptic feedback.

The processing unit compares the most recent stride data with one or more target patterns. Such exemplary system may use a means of extracting a standardized section of the time series. The reference trigger that identifies a new gait cycle is, for example, the first point during a small linear and angular acceleration period recorded by the shoe IMU during gait.

The feedback provided is related to the error between the measured knee angle and a preferred gait cycle similar to a normalized walking speed. The target gait pattern for training may be chosen based on subject-specific measurements and calculation of the normalized speed.

The gait cycle is split as follows: 0% represents the heel strike event; 50-90% gait cycle represents the ‘flexion’ feedback phase; and the remainder of the cycle which is 0-50% and 90-100% represent the ‘extension’ feedback phase. The feedback signals overall are driven by measures of errors between the target and measured signals. The DTW-warped target and measured signals are used as the basis of the error calculations.

Two error metrics are calculated for each phase: The mean absolute error and the sign of the mean signed error. Essentially the needle deflections are set by a quantity akin to the product of the (sign of the mean signed error) and (the mean absolute error).

A feedback display is shown in FIG. 1b. The left side shows extension error feedback and the right side shows flexion error feedback needle. Pictographic symbols represent ‘too flexed’ or ‘too extended’ relative to the target. Three point zones are used for scoring based on the needle angles.

In one instance, the subject is afflicted with hemiplegic cerebral palsy has increased flexion at initial contact which causes the extension phase feedback needle to indicate excessive flexion and guide the subject to extend the knee more during this phase. In this case, the flexion phase needle indicates that the gait pattern is normal.

In another instance, the subject is afflicted with cerebral palsy or recently had a stroke and shows a gait pattern with generalized decrease in knee movement in which (1) knee flexion increases during the extension phase which causes the extension phase feedback needle to indicate excessive flexion and guide the subject to extend the knee during this phase and (2) knee flexion decreases during the flexion phase which causes the flexion phase needle to guide the subject to bend the knee more during this phase.

In an additional instance, the subject is afflicted with hemiplegia and shows a gait pattern which has low knee flexion in the flexion phase which results in (1) a normal knee flexion pattern during the extension phase which causes the extension phase feedback needle to be in the point scoring range, reinforcing this phase of the pattern and (2) decreased knee flexion during the flexion phase, which causes the flexion phase needle to guide the subject to bend the knee more during this phase.

In another instance, the subject is afflicted with cerebral palsy and shows a gait pattern which has excessive flexion in the extension phase, which results in (1) the knee pattern being excessively flexed during most of the gait cycle, which causes the extension phase feedback needle to indicate excessive flexion and guide the subject to extend the knee more during this phase to normalize the gait pattern.

In a further embodiment and as shown in FIG. 1a gait retraining is performed on a treadmill with inertial sensor setup on a participant's paretic lower limb. FIG. 1b shows the feedback interfaces: the left panel: extension period; right panel: flexion period. If the needle (the pointed line) is on the top half of the interface, it indicates the current pattern is more flexed than the target. If the needle is on the bottom half of the interface, it indicates the current pattern is more extended than the target. The needle moves closer to the color point zone when the measured knee flexion pattern matches the target knee flexion pattern more closely. In this gray scale figure, the color point zone is shown as the shaded area inside of circle with B indicating the color blue, Y indicating the color yellow and G indicating the color green. The tick marks on the right side of the circle delimit the maximum range. Motivation was provided by awarding of points which were added to a cumulative score (1, 2, 3 points according to blue, yellow, green sectors of the target color zone). In this frame, the score would increase if the participant increase the knee flexion in the swing period towards the target.

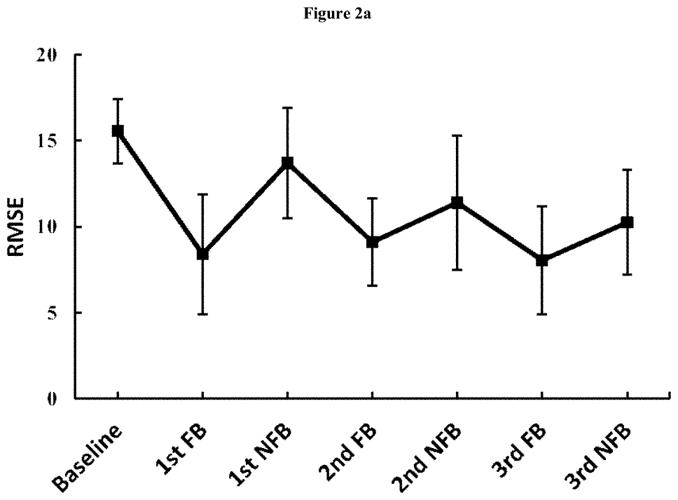

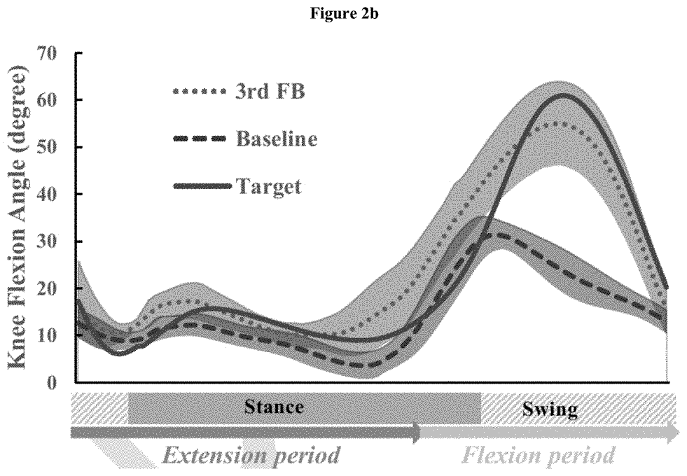

In a further embodiment and as shown in FIGS. 2a and 2b, data points were averaged from the last 10 strides of each trial. FIG. 2a shows the mean and SD (error bar) of knee flexion angle RMSE over repeated training bouts. FIG. 2b shows mean and SD (shaded band) of knee flexion.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### The Development of a Real-Time Pattern-Based Kinematic Feedback Gait Retraining Platform

Rehabilitative biofeedback motor training enhances task-specific practice by augmenting disrupted native feedback, thereby promoting modifications to feedforward control strategies and reduction of variability in performance. To motivate children's motor learning with precise cues towards targets, a wearable sensor based visual kinematics feedback platform was developed and it was found that the maximum knee flexion patterns of typically developing children were redirected to the target patterns in response to the feedback. The hardware requirements and setup time of the feedback platform are much lower than those for motion analysis system or virtual reality environments. The platform works with conventional treadmill or overground walkway, which is also suitable for community or home setup. The feedback prototype described herein provided a feedback signal driven by the error across the whole gait cycle. To provide individuated training to various deficit types in different gait phases, a prototype was developed to isolate the feedback according to extension and flexion roles in the gait cycle. After further development, the feedback gait retraining platform was tested on a pediatric patient with cerebral palsy (CP).

Materials and Methods:

In this feedback gait retraining platform, three inertial sensors (MTW Devkit, Xsens Technologies BV, Enschede, Netherlands) that measure three-dimensional segment accelerations and orientations during gait were placed on the participant's paretic lower limb (FIG. 1a). The thigh sensor was located at the midpoint of the line from the patella to the anterior superior iliac spine and the sensor's long axis was perpendicular to the line. The shank sensor was placed on the most prominent section of the gastrocnemius parallel to the thigh; the sensor's orientation was adjusted with the assistance of the MTW software to match the yaw and pitch rotations of the thigh sensor. The knee flexion angle was defined as the difference between the thigh and shank sensors' roll rotation (the rotation on longitudinal axis). The foot contact was detected by the heel sensor taped to the heel of the shoe with the sensor's long axis vertical to the ground at standing posture. Based on real-time processing of data collected from the sensors, the platform provides concurrent kinematic feedback on a computer monitor. The feedback calculation and interface are developed by the study team using MATLAB (The Mathworks, Natick, Mass.) and Xsens programs.

The target knee flexion pattern was selected from typically developing children's patterns based on participant's speed height ratio. All analyzed gait cycles were normalized to 101 samples along the temporal axis. Dynamic time warping, a method for similarity measurement between two temporal sequences, was used to generate two warped signals to attenuate temporal offsets between the measured and the target knee flexion patterns. The feedback on the knee flexion angle was then divided by two periods that the extension or the flexion plays the primary role in a gait cycle. On the feedback interface (FIG. 1b), the left panel showed the participant's extension period that includes the late swing, early and mid-stance phases of a gait cycle; the right panel showed the participant's flexion period that includes the terminal stance, early and mid-swing phases of a gait cycle. The knee flexion pattern for the most recent stride was compared with a target pattern. The needle positions were governed by average and signed error metrics computed over the two periods. A quadratic relationship between the needle angle y and error measure x was defined as

|y|==ax²−c (1)

Three color point zones (FIG. 1b) were defined by the needle angles that: if |y|<y_{3P}, 3 point was recorded; if y_{3P}<|y|<y_{2P}, 2 point was recorded; if y_{2P}<|y|<y_{1P}, 1 point was recorded. An x-axis zero crossing was defined by an adjustable parameter v to control the error below threshold.

Data from a participant with right hemiplegic CP (male, age 11, height 1.55 m, weight 46.3 kg) was collected to test the feedback gait retraining platform. The participant presented stiff knee gait pattern on his paretic side (right), with insufficient knee flexion in the mid-swing phase. The knee angles measured by the inertial sensors were calibrated by registering the angles measured at two standing postures (full extension and 60 degree flexion of the knee) to the ones measured by goniometers. After calibration, the participant selected a comfortable speed (0.54 m/s) for treadmill walking and this speed was then used as the training speed. The participant walked on the treadmill for 1 minute while his knee flexion pattern in natural walking was recorded as the baseline data. The participant then experienced a single session of gait retraining which included repeated (three) training bouts of 3-minute Feedback (FB) and 3-minute Non-Feedback (NFB) trials. Before the first training bout, the feedback interface was introduced to the participant and the participant practiced walking with feedback for 1 minute. The difference between the FB and NFB trials is that only in the FB trials a computer monitor displayed the real-time feedback of the knee flexion pattern of the participant. In the beginning of each FB trial, the participant was told to follow the cue on the feedback interface and try to modify his knee flexion pattern to rotate the needles towards the targets. In the beginning of each NFB trial, the participant was told to recall his walking pattern in the FB trial and try to maintain it. The training bouts were separated by 3-minute seated rest to avoid fatigue.

The root-mean-square error (RMSE) between the measured and target knee flexion angles was calculated in the last ten strides of the baseline, 1^{st} FB, 1^{st} NFB, 2^{nd} FB, 2^{nd} NFB, 3^{rd} FB, and 3^{rd} NFB trials. The average RMSE across the ten strides in each trial was then compared between trials (FIG. 2a). The mean knee flexion angle curves of the last ten strides in the baseline and 3rd FB trials were compared with the target knee flexion angle curve (FIG. 2b).

Feedback was provided in two phases of the cycle, so that users are rewarded (with points) for appropriate bending and straightening. Additionally, additional processing was employed to generate the feedback signals to make them more forgiving of small timing variations and more reflective of the overall pattern similarity. The XsensMTw sensors used have 3D orientation information which comes from: (1) magnetometers which sense heading relative to magnetic north, (2) accelerometers which sense orientation relative to vertical, (3) gyroscopes which sense angular velocity. The magnetometer signal is susceptible to drift; this issue was worked around by relying only on the accelerometer and gyroscope based readings. This works because the characteristic movement (knee movement during walking) is in a plane.

Results:

Reduction in knee flexion angle RMSE relative to baseline was observed in all trials with and without feedback (FIG. 2a). Adaptation to extension and flexion period error-based feedback showed increased swing phase knee flexion and maintenance of appropriate extension through majority of stance (FIG. 2b).

Discussion:

A method for developing real-time pattern-based kinematic feedback gait retraining platform was developed. The method provided specific feedback in separate extension and flexion roles across the gait cycle. The case study on a child with hemiplegic CP showed that the participant increased swing phase knee flexion while maintained stance phase extension accordingly to the feedback cues. The participant's knee pattern was prominently improved in the first feedback trial and retained without feedback, which indicated that the real-time visual feedback gait retraining is promising in correcting and refining specific motor patterns in terms of individualized training.

### Example 2

### Two Needle Gait Feedback

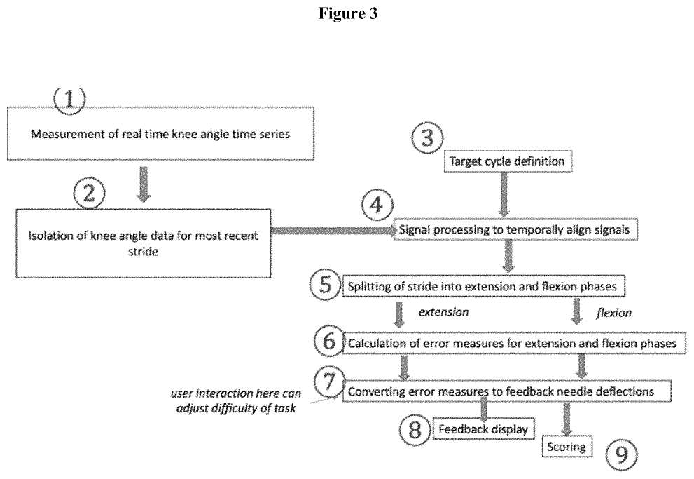

The system described in this example processes signals from 3 inertial sensors (IMU) placed on one limb and provides two feedback needles and scoring functionality. The system can also be run in a measurement mode. FIGS. 3-6 are related to this example.

Measurement of Real Time Angle Time Series:

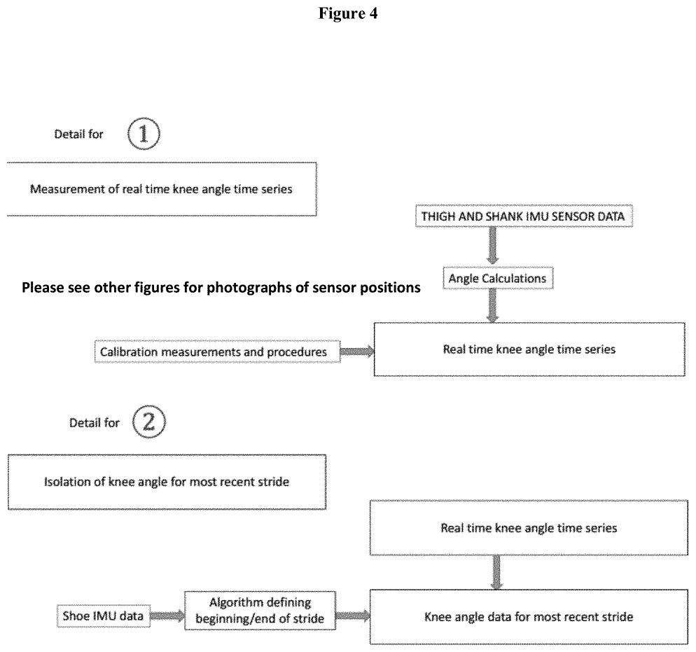

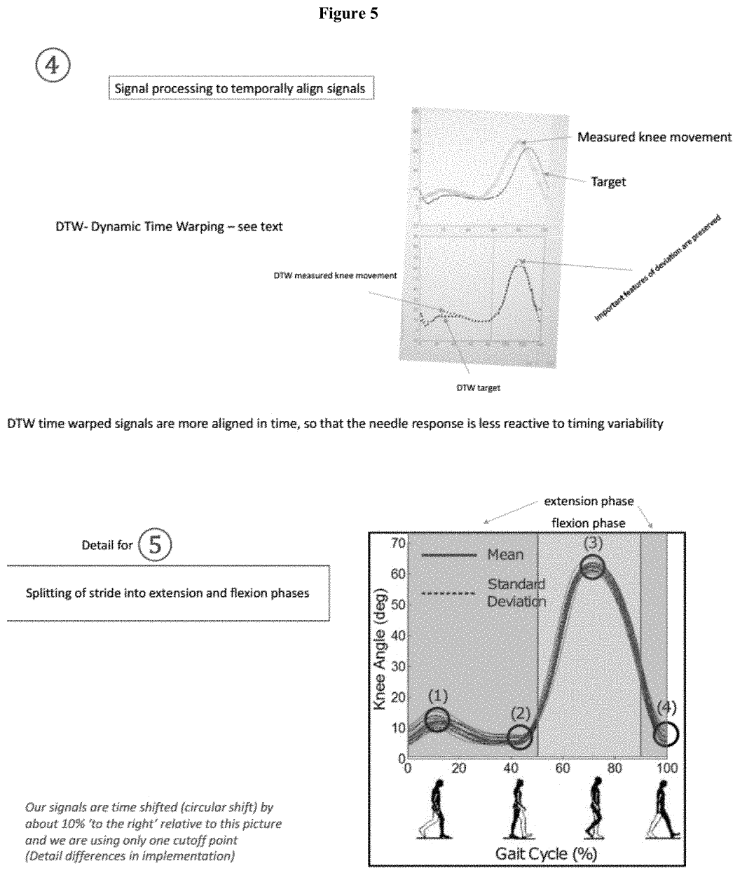

This feedback method measures one anatomical angle, specifically, knee flexion angle. Either the right or left leg must be chosen. This angle is measured in real time using two inertial measurement units (IMUs) placed on the leg (See FIG. 4).

IMU Based Knee Flexion Angle:

The knee angle measured by the IMUs is defined as the difference between the thigh and shank sensors' ‘Roll’ rotation. Each sensor's ‘Roll’ is defined as the rotation on the longitudinal axis of the sensor. The thigh sensor is located on the anterior aspect of the thigh midway from the patella to the anterior superior iliac spine (ASIS); it is longitudinally oriented perpendicularly to this line. A second sensor is placed on the posterior aspect of the shank on the most prominent section of the gastrocnemius parallel to the thigh; this sensor is placed with the assistance of the MTW software to match the ‘Yaw’ and ‘Pitch’ rotations on both sensors.

Calibration and Scaling from IMU-Based Angles to Anatomical Knee Angles:

Briefly, a long arm goniometer (Lafayette Gollehon Extendable Goniometer) is used to perform a two point calibration to relate IMU measured knee angle to an anatomical knee angle. This is because the IMU referenced angle depends on many things such as anatomical shape and exact positioning on the leg. Other scaling has also been used to relate the measurements to the target pattern described below.

Isolation of Knee Angle Data for Most Recent Stride:

The feedback is on the most recent stride and is compared with a target pattern. Therefore, a means of extracting a standardized section of the time series is needed. This is done based on an event that can be detected in every stride as follows. The third IMU attached to the heel of the shoe is used for this (FIG. 1a).

Description of Event Defining Beginning and End of Stride:

(The x/y/z references below are relative to the sensor package). The reference trigger that identifies a new gait cycle is the first point during a small linear acceleration and angular velocity period (‘silence’) recorded by the shoe IMU during gait. Linear accelerations are in the units of meters per second squared and angular velocities are in the units of degrees per second in the following. The ‘silence’ period is detected when 5 consecutive samples follow 4 concurrent conditions: (1) the linear x acceleration is greater than 8.31; (2) the linear x acceleration is less than 11.31; (3) the angular y velocity is greater than −0.2; (4) the angular y velocity is less than 0.8. This point occurs shortly after heel contact, when the foot establishes full contact with the floor.

Target Cycle Definition:

The feedback provided is related to the error between the measured knee angle (most recent stride) and a preferred gait cycle. This gait cycle is extracted from a published database and is referenced to the ratio of walking speed and subject height (v/h). Consider this as a normalized walking speed—‘body heights per second etc.’ ‘Normative’ flexion patterns vary with the normalized walking speed. The target cycles used were based on an article by Bovi²⁹. The source populations are n=20, ages 6-17; mean 10.8±3.2, mass=41.4±15.5 kg, and 9 males, 11 females. All the walking trials were divided into one of the following four classes based on walking speed (m/s) normalized to subject's height (m) (v/h): very slow (XS) for v/h<0.6; slow (S) for 0.6<v/h<0:8; medium (M) for 0.8<v/h<1; fast (L) for v/h>1. The target gait pattern for training is chosen based on subject-specific measurements and calculation of the normalized speed.

Signal Processing to Temporally Align Signals (FIG. 5):

During the development process it was discovered that there was a need to make the feedback less sensitive to small variations in timing. These could be from uncertainty in the trigger timing or from true variation in the gait pattern. As a first stage of processing for signal comparison the Dynamic Time Warping (DTW) algorithm³⁰ is used to ‘align’ the target and measured signals. In time series analysis, dynamic time warping (DTW) is an algorithm for measuring similarity between two temporal sequences. These sequences may vary in speed. For instance, similarities in walking may be detected using DTW, even if one person was walking faster than the other, or if there were accelerations and decelerations during the course of an observation. DTW has been applied to temporal sequences of video, audio, and graphics data. Any data that can be turned into a linear sequence can be analyzed with DTW. Both the target and measured signals are initially resampled to 101 points. The DTW error metric is not used directly, instead a warping path is determined from a list of indices into each source signal. Using that warping path separate error metrics are calculated for the flexion and extension phases indicated below.

Splitting of Stride into Extension and Flexion Phases: (FIG. 5)

An early prototype had a single feedback signal for the whole gait pattern. However, after observing the difficulties in using the earlier prototype to train the gait pattern, ‘assistive’ cues were added for the timing and amount of the peak knee flexion during the swing phase. These particular cues were to some extent driven by the fact that a novel gait pattern was being trained that changed the peak knee flexion in the swing phase.

It was found that children tended to adopt a change all the way through the cycle. A ‘More flexed swing phase’ cue led to an adaptation of more flexed gait all through the pattern. The children were training more on the assistive cues than the overall needle cue. Applicants conceived that the cycle should be broken into two phases and feedback should be provided using two needles. These were first conceived as ‘stance’ and ‘swing’ phases, because this is the way the gait cycle is traditionally broken up.

Looking at the right leg, the “stance” is when the right leg is on the ground and the “swing” is when it is off the ground. We are only considering one leg in this instance, not the interaction with the other leg.

Applicants determined that final changes were necessary after the surprising realization that it is easier to respond to the feedback if the cycle is instead split in a way that reflects more characteristic periods of the limb positioning. There is a phase in which the limb is more flexed, and a phase in which it is more extended. If the phases inherently reflect transitions (as in the stance and swing phases used first) the ‘more flexed’ ‘more extended’ cues do not work well.

Transition Points for Extension and Flexion Phases:

Functionally and for replication purposes what is below represents the algorithm accurately. The gait cycle is split as follows: 0% represents the heel strike event (this is standard for gait analysis); 50-90% gait cycle represents the ‘flexion’ feedback phase; and the remainder of the cycle which is 0-50% and 90-100% represent the ‘extension’ feedback phase.

Calculation of Error Measures for Extension and Flexion Phases:

The feedback signals overall are driven by measures of errors between the target and measured signals. This section explains how these are computed for the extension and flexion phases defined above.

Calculation from DTW-Processed Signals:

The DTW-warped target and measured signals are used as the basis of the error calculations. The splitting into phases requires a way to correctly reference the relevant portions of the warped signals. This is done by reference to the target signal. The 50% and 90% cutoff points in both target and measured signals are referenced to the position in the target signal.

Overview of how the needle deflections are derived: Two error metrics are calculated for each phase: The mean absolute error and the sign of the mean signed error. For the mean absolute error, the investigators look pointwise across the cycle through the DTW warped signals and calculate the absolute error for each point. This is the absolute value of the difference between the values at each point in the cycle. The investigators then take the mean by summing and dividing by the number of points. If the measured pattern is more flexed than the target by a constant amount, this gives rise to a value for mean absolute error, and if it is less flexed by the same constant amount this gives rise to the same value. This metric is therefore sensitive to pattern deviation but cannot inform the direction of the deviation.

The sign of the mean signed error is similar to the mean absolute error, but the signed error is being taken at this point. For a case in which the measured knee angle is consistently (or on average) exceeding the target, this signal will be positive. The Sign of this quantity (also known as signum function) is only being used. Essentially the needle deflections are set by a quantity akin to the product of the (sign of the mean signed error) and (the mean absolute error). This captures the overall deviation, and the sense of the overall deviation. It works better than simply using the mean absolute error (no direction sense) or the mean signed error (sections over and under the target angle self-cancel).

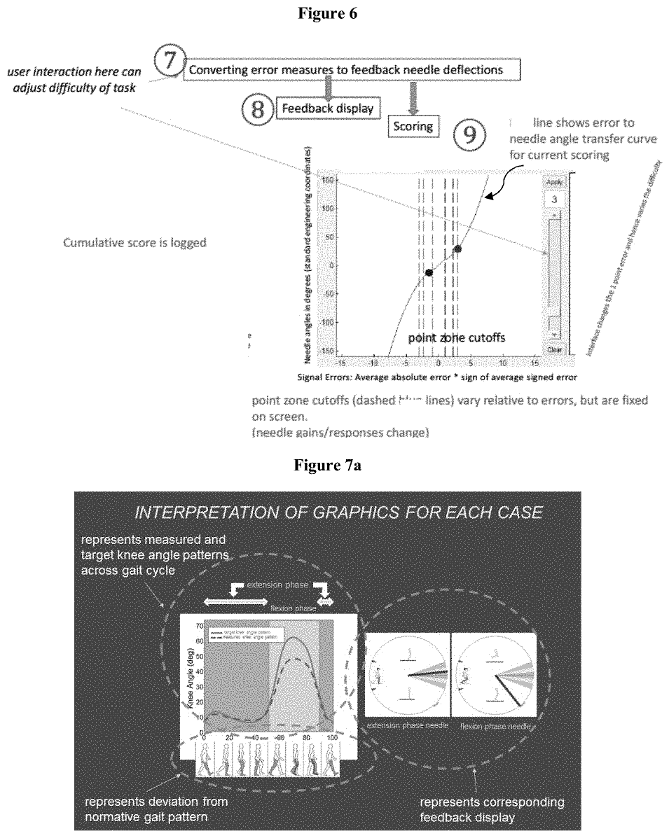

Converting Error Measures to Feedback Needle Deflections: (FIG. 6)

This section describes how the error metrics map to the needle responses. It is easiest to understand with reference to the above sections and FIGS. 3-5.

Description of Conversion:

FIG. 6 shows the transfer curve between errors and needle positions, with the x axis being the product ‘Average absolute error * sign of average signed error’ as discussed above. Two needle angle variables are defined, one for each needle: (a) Needle deflections are 0 degrees at 3 o'clock position; (b) Positive 90 degrees=12 o'clock; negative 90=6 o'clock; (c) Maximum deflection=+/−160 degrees, matching the tick marks. FIG. 6 shows a quadratic mapping with a linear connecting section at the center. This allows more range for larger errors but better sensitivity near the target range. The curve is user-adjustable. The vertical dashed lines define the scoring.

Feedback Display (FIG. 1b):

Left side: Extension error feedback; Right side: Flexion error feedback needle; The pictographic symbols represent ‘too flexed’, ‘too extended’ relative to the target. For instance, a gait with excessive flexion in the flexion period swing will register with the right side needle in the top half of the screen.

Scoring:

Three point zones are defined. The scoring is fixed relative to the needle angles. The difficulty is varied if needed by varying the needle response. This was chosen in preference to widening or narrowing the sectors visually, which was hypothesized to be more transparent to a child experiencing difficulty with the task. The slider (value 3 shown) varies the absolute error value that maps to the outer 1 point cutoff. When the slider is manipulated, the response curve changes. The other ⅔ point cutoffs are calculated/scaled in by appropriate parameter selection for the curve. If the 1 point cutoff value is lowered: (a) a lower error value will be required to score 1 point; (b) the task will seem ‘more difficult’; (c) the overall response will feel ‘more sensitive’ (gain/slope of response curve is higher); and (d) a higher total range of errors will be displayable before overranging the needles.

If the 1 point cutoff value is increased: (a) a higher error value will be required to score 1 point; (b) the task will seem ‘less difficult’; (c) the overall response will feel ‘less sensitive’ (gain/slope of response curve is lower), and (d) a lower total range of errors will be displayable before overranging the needles.

### Example 3

### Gait Patterns and Feedback Provided

This example provides gait patterns a subject may have and examples of feedback that may be provided to the subject in response to these gait patterns. This feedback is expected to provide treatment to the subject by allowing for the subject to correct his or her gait based on data provided by the system of this invention.

FIG. 7b shows a gait pattern with increased flexion at initial contact. Whereas in the normative gait pattern the knee is almost fully extended at initial contact (0% gait cycle), in this pattern the subject's knee remains bent. In the feedback displays, the extension phase feedback needle is indicating excessive flexion and guiding the subject to extend the knee more during this phase, whereas the cue from the flexion phase needle is indicating that the pattern here is normative. This gait pattern is seen in some children with hemiplegic cerebral palsy.

FIG. 7c shows a gait pattern with generalized decrease in knee movement. The overall active range of motion of the knee is reduced. There is increased knee flexion during the extension phase and decreased knee flexion during the flexion phase. The extension phase feedback needle is indicating excessive flexion and guiding the subject to extend the knee during this phase, whereas the flexion phase needle is guiding the subject to bend the knee more during this phase. This pattern is sometimes observed in the gait of people with cerebral palsy or following stroke.

FIG. 7d shows a gait pattern which has low knee flexion in the flexion phase, also known as stiff knee gait. The knee flexion pattern is normative during the extension phase and shows decreased knee flexion during the flexion phase. The extension phase feedback needle is in the point scoring range, reinforcing this phase of the pattern, whereas the cue from the flexion phase needle is guiding the subject to bend the knee more during this phase. This gait pattern is associated with hemiplegia (stroke or cerebral palsy) and is often accompanied by other compensations needed for foot clearance during the swing phase.

FIG. 7e shows a gait pattern which has excessive flexion in extension phase, also known as crouch gait. In this pattern, the knee pattern is excessively flexed during most of the gait cycle, however the maximum knee flexion is close to normative. The extension phase feedback needle indicates excessive flexion and is guiding the subject to extend the knee more during this phase to normalize the gait pattern. In the flexion phase there is also need for moderate correction to address deviation in this phase. This gait pattern is associated with cerebral palsy and relates to lower extremity spasticity.

FIG. 7f shows a normative gait pattern in which the measured knee flexion pattern is similar to the normative pattern. In this case, both needles provide feedback in the point scoring range, reinforcing the maintenance of the pattern.

### REFERENCES

[1] M. W. Brault, Americans with disabilities: 2010, Current population reports, US Census Bureau, Washington, D C, 2012, pp. 0-131.
[2] M. Brown, W. A. Gordon, Impact of impairment on activity patterns of children, Arch. Phys. Med. Rehabil. 68(12) (1987) 828-32.
[3] R. Jahnsen, L. Villien, G. Aamodt, J. K. Stanghelle, I. Holm, Musculoskeletal pain in adults with cerebral palsy compared with the general population, J. Rehabil. Med. 36(2) (2004) 78-84.
[4] D. R. Carter, B. Tse, The pathogenesis of osteoarthritis in cerebral palsy, Dev. Med. Child Neurol. 51 (2009) 79-83.
[5] N. Paneth, T. Hong, S. Korzeniewski, The descriptive epidemiology of cerebral palsy, Clin. Perinatol. 33(2) (2006) 251-267.
[6] N.S.C.I.S. Center, 2014 Annual Statistical Report for the Spinal Cord Injury Model Systems Public Version, (2014).
[7] W. A. Bauman, The potential metabolic consequences of cerebral palsy: inferences from the general population and persons with spinal cord injury, Dev. Med. Child Neurol. 51(s4) (2009) 64-78.
[8] D. H. Sutherland, J. R. Davids, Common gait abnormalities of the knee in cerebral palsy, Clin. Orthop. Relat. Res. 288 (1993) 139-147.
[9] T. A. Wren, S. Rethlefsen, R. M. Kay, Prevalence of specific gait abnormalities in children with cerebral palsy: influence of cerebral palsy subtype, age, and previous surgery, J. Pediatr. Orthop. 25(1) (2005) 7983.
[10] A. Nieuwenhuys, S. Ounpuu, A. Van Campenhout, T. Theologis, J. De Cat, J. Stout, et al., Identification of joint patterns during gait in children with cerebral palsy: a Delphi consensus study, Dev. Med. Child Neurol. 58(3) (2016) 306-313.
[11] P. Krawetz, P. Nance, Gait analysis of spinal cord injured subjects: effects of injury level and spasticity, Arch. Phys. Med. Rehabil. 77(7) (1996) 635-638.
[12] P. A. DeLuca, S. Ounpuu, R. B. Davis, J. H. Walsh, Effect of hamstring and psoas lengthening on pelvic tilt in patients with spastic diplegic cerebral palsy, J. Pediatr. Orthop. 18(6) (1998) 712-718.
[13] N. Smania, P. Bonetti, M. Gandolfi, A. Cosentino, A. Waldner, S. Hesse, et al., Improved gait after repetitive locomotor training in children with cerebral palsy, Am. J. Phys. Med. Rehabil. 90(2) (2011) 137-149.
[14] K. A. Leech, C. R. Kinnaird, C. L. Holleran, J. Kahn, T. G. Hornby, Effects of Locomotor Exercise Intensity on Gait Performance in Individuals With Incomplete Spinal Cord Injury, Phys. Ther. 96(12) (2016) 19191929.
[15] D. Schliessmann, C. Schuld, M. Schneiders, S. Derlien, M. Glockner, T. Gladow, et al., Feasibility of visual instrumented movement feedback therapy in individuals with motor incomplete spinal cord injury walking on a treadmill, Front. Hum. Neurosci. 8 (2014) 416.
[16] D. W. Rha, K. Cahill-Rowley, J. Young, L. Torburn, K. Stephenson, J. Rose, Biomechanical and Clinical Correlates of Stance-Phase Knee Flexion in Persons With Spastic Cerebral Palsy, PMR 8(1) (2016) 11-8.
[17] J. A. Barrios, K. M. Crossley, I. S. Davis, Gait retraining to reduce the knee adduction moment through real-time visual feedback of dynamic knee alignment, J. Biomech. 43(11) (2010) 2208-2213.
[18] L. Donovan, M. A. Feger, J. M. Hart, S. Saliba, J. Park, J. Hertel, Effects of an auditory biofeedback device on plantar pressure in patients with chronic ankle instability, Gait Posture 44 (2016) 29-36.
[19] P. B. Shull, K. L. Lurie, M. R. Cutkosky, T. F. Besier, Training multi-parameter gaits to reduce the knee adduction moment with data-driven models and haptic feedback, J. Biomech. 44(8) (2011) 1605-1609.
[20] S. J. Sober, P. N. Sabes, Flexible strategies for sensory integration during motor planning, Nat. Neurosci. 8(4) (2005) 490-497.
[21] M. L. Peterson, E. Christou, K. S. Rosengren, Children achieve adult-like sensory integration during stance at 12-years-old, Gait Posture 23(4) (2006) 455-463.
[22] R. M. Rine, K. Rubish, C. Feeney, Measurement of sensory system effectiveness and maturational changes in postural control in young children, Pediatr. Phys. Ther. 10(1) (1998) 16-22.
[23] K. J. Sullivan, S. S. Kantak, P. A. Burtner, Motor Learning in Children: Feedback Effects on Skill Acquisition, Phys. Ther. 88(6) (2008) 720-732.
[24] R. Sigrist, G. Rauter, R. Riener, P. Wolf, Augmented visual, auditory, haptic, and multimodal feedback in motor learning: a review, Psychonomic bulletin & review 20(1) (2013) 21-53.
[25] N. S. Hamed, M. S. Abd-elwahab, Pedometer-based gait training in children with spastic hemiparetic cerebral palsy: a randomized controlled study, Clin. Rehabil. 25(2) (2011) 157-65.
[26] L. van Gelder, A. T. Booth, I. van de Port, A. I. Buizer, J. Harlaar, M. M. van der Krogt, Real-time feedback to improve gait in children with cerebral palsy, Gait Posture 52 (2016) 76-82.
[27] J. C. van den Noort, F. Steenbrink, S. Roeles, J. Harlaar, Real-time visual feedback for gait retraining: toward application in knee osteoarthritis, Medical & biological engineering & computing 53(3) (2015) 275-86.
[28] Oliveira N, Ehrenberg N, Cheng J F, Blochlinger S, Barrance P. 2017. Knee flexion modifications during pediatric gait using visual feedback. Proceedings of the 41st Annual Meeting of the American Society of Biomechanics, Boulder, Colo., USA, Aug. 8-11, 2017.
[29] Bovi G, Rabuffetti M, Mazzoleni P, Ferrarin M. 2011. A multiple-task gait analysis approach: Kinematic, kinetic and EMG reference data for healthy young and adult subjects. Gait Posture. 33:6-13).
[30] Sakoe H, Chiba, S. 1978. Dynamic programming algorithm optimization for spoken word recognition. IEEE Trans Acoust. 26:43-49.

It is to be understood that the invention is not limited to the particular embodiments of the invention described above, as variations of the particular embodiments may be made and still fall within the scope of the appended claims.

The invention is further described by the following numbered paragraphs:
1. A system for biofeedback of gait training of a subject in need thereof, comprising:
   a. sensors configured to be releasably attachable to at least one leg of the subject, said sensors configured to provide leg positional information during at least motion of the at least one leg during a walking motion of the at least one leg;
   b. a processing unit for receiving the positional information from the sensors and for generating data indicative of knee flexion angles and knee extension angles during at least the walking motion of the at least one leg; and for generating corresponding respective knee flexion and knee extension angles training signals based on the generated data;
   c. at least one feedback unit configured to receive the generated training signals to provide feedback to the subject during at least the walking motion of the at least one leg, the feedback including at least a first component indicative of the subject's knee flexion angles during at least the walking motion of the at least one leg, and a second component indicative of the subject's knee extension angles during at least the walking motion of the at least one leg.
2. The system of paragraph 1 wherein the processing unit is further configured to generate the training signals indicative of knee flexion angles relative to corresponding predetermined knee flexion reference angles.
3. The system of any of paragraphs 1-2 wherein the processing unit is further configured to generate the training signals indicative of knee extension angles relative to corresponding predetermined knee extension reference angles.
4. The system of any of paragraphs 1-3, wherein the at least one feedback unit is at least one of a display, projector, sound generator or haptic feedback device.
5. The system of any of paragraphs 1-4 further comprising a treadmill positioned in proximity to the feedback device.
6. The system of any of paragraphs 1-5, wherein said sensors comprise at least one first, second and third sensors configured to be releasably attachable to respective one's of thigh, shank and heel of the at least one leg of the subject.
7. The system of any of paragraphs 1-6, wherein said sensors comprise at least one of inertial, magnetic, electromagnetic, micro-electromechanical, or optical circuits configured to sense leg position.
8. A method of biofeedback for gait training of a subject in need thereof, comprising the steps of:
   a. releasably attaching sensors to at least one leg of the subject, said sensors being configured to provide leg positional information during at least motion of the at least one leg during a walking motion of the at least one leg;
   b. transmitting by the sensors to a processing unit, leg positional information during at least motion of the at least one leg during a walking motion of the at least one leg;
   c. generating by the processing unit based on the leg positional information, data indicative of knee flexion angles and knee extension angles during at least the walking motion of the at least one leg;
   d. generating by the processing unit, respective knee flexion and knee extension training signals based on the generated data;
   e. at least one feedback unit receiving by at least one feedback unit, the generated training signals; and
   f. providing feedback by the at least one feedback unit during at least the walking motion of the at least one leg based on the training signals, the feedback including at least a first component indicative of the subject's knee flexion angles during at least the walking motion of the at least one leg, and a second component indicative of the subject's knee extension angles during at least the walking motion of the at least one leg.
9. The method of paragraph 8 wherein the step of generating the training signals generates training signals indicative of knee flexion angles relative to corresponding predetermined knee flexion reference angles.
10. The method of any of paragraph 8-9 wherein the step of generating the training signals generates training signals indicative of knee extension angles relative to corresponding predetermined knee extension reference angles.
11. The method of any of paragraphs 8-10 wherein the step of feedback generating step generates feedback of at least one of a display, projector, sound generator or haptic feedback.
12. The method of any of paragraphs 8-11 further comprising subject walking on a treadmill positioned in proximity to a device providing the feedback.
13. The method of any of paragraphs 8-12, wherein the step of releasably attaching sensors to at least one leg of the subject further comprises releasably attaching the sensors to respective one's thigh, shank and heel of the at least one leg of the subject.
14. A system for biofeedback for gait training of a subject in need thereof, comprising sensors capable of being put on the thigh, shank and heel of the subject, and two separate output displays, the system being capable of measuring knee flexion data and knee extension data wherein a first output display shows knee flexion data and a second output display shows knee extension data in real time.
15. A method of biofeedback for gait training of a subject in need thereof, comprising the steps of:
   a. applying sensors to the thigh, shank and heel of the subject such that the sensors are capable of measuring data needed to calculate knee flexion data and knee extension data;
   b. outputting biofeedback data in two separate output displays, a first output display showing knee flexion data and a second output display showing knee extension data;
   c. instructing the subject to adapt gait in real time based on feedback data.
16. The system of paragraph 14 or the method of paragraph 15, wherein the subject is afflicted with a gait disorder, cerebral palsy (CP), or hemiplegic CP.
17. The system or method of any one of paragraphs 14-16, wherein the subject is less than 18 years old, less than 16 years old, less than 14 years old less than 12 years old, or greater than 7 years old and less than 16 years old.
18. The system or method of any one of paragraphs 14-17, wherein the subject's gait pattern has
   a. increased flexion at initial contact of the gait cycle compared to a normative gait pattern;
   b. a generalized decrease in knee movement compared to a normative gait pattern;
   c. a low knee flexion in the flexion phase of the gait cycle compared to a normative gait pattern;
   d. an excessive flexion in extension phase of the gait cycle compared to a normative gait pattern; or
   e. an insufficient knee flexion in the mid-swing phase compared to a normative gait pattern.
19. The system or method of any one of paragraphs 14-18, wherein the sensors are inertial sensors.
20. The system or method of any one of paragraphs 14-19, wherein the sensors measure or are capable of measuring three-dimensional segment accelerations and/or orientations during gait.
21. The system or method of any one of paragraphs 14-20, wherein the thigh sensor is located or to be located at the midpoint of the line from the patella to the anterior superior iliac spine and the thigh sensor's long axis is perpendicular to the line.
22. The system or method of any one of paragraphs 14-21, wherein the shank sensor is located or to be located on the most prominent section of the gastrocnemius parallel to the thigh.
23. The system or method of any one of paragraphs 14-22, wherein the knee extension data comprises data concerning the subject's late swing, early, and mid-stance phases of a gait cycle.
24. The system or method of any one of paragraphs 14-23, wherein the knee flexion data comprises data concerning the subject's terminal stance, early and mid-swing phases of a gait cycle.
25. The system or method of any one of paragraphs 14-24, wherein the first output display comprises a flexion error feedback needle.
26. The system or method of any one of paragraphs 14-25, wherein the second output display comprises an extension error feedback needle.
27. The system or method of any one of paragraphs 14-26, wherein the biofeedback is less sensitive to small variations in timing.
28. The method of any one of paragraphs 15-27, wherein the method is performed periodically.
29. The method of any one of paragraphs 15-28, wherein the method is performed periodically at least once per week, at least once every two weeks, at least once a month, at least once every 3 months, at least once every 6 months, at least once every year.

We claim:

1. A system for biofeedback of gait training of a subject in need thereof, comprising:
a. sensors configured to be releasably attachable to at least one leg of the subject, said sensors configured to provide leg positional information during at least motion of the at least one leg during a walking motion of the at least one leg;
b. a processing unit for receiving the positional information from the sensors and for generating data indicative of knee flexion angles and knee extension angles during at least the walking motion of the at least one leg; and for generating corresponding respective knee flexion and knee extension angles training signals based on the generated data;
c. at least one feedback unit configured to receive the generated training signals to provide feedback to the subject during at least the walking motion of the at least one leg, the feedback including at least a first component indicative of the subject's knee flexion angles during at least the walking motion of the at least one leg, and a second component indicative of the subject's knee extension angles during at least the walking motion of the at least one leg.

2. The system of claim 1 wherein the processing unit is further configured to generate the training signals indicative of knee flexion angles relative to corresponding predetermined knee flexion reference angles.

3. The system of claim 1 wherein the processing unit is further configured to generate the training signals indicative of knee extension angles relative to corresponding predetermined knee extension reference angles.

4. The system of claim 1, wherein the at least one feedback unit is at least one of a display, projector, sound generator or haptic feedback device.

5. The system of claim 1 further comprising a treadmill positioned in proximity to the feedback device.

6. The system of claim 1, wherein said sensors comprise at least one first, second and third sensors configured to be releasably attachable to respective one's of thigh, shank and heel of the at least one leg of the subject.

7. The system of claim 1, wherein said sensors comprise at least one of inertial, magnetic, electromagnetic or optical circuits configured to sense leg position.

8. The system of claim 1, wherein said sensors are capable of measuring three-dimensional accelerations.

9. A method of biofeedback for gait training of a subject in need thereof, comprising the steps of:
a. releasably attaching sensors to at least one leg of the subject, said sensors being configured to provide leg positional information during at least motion of the at least one leg during a walking motion of the at least one leg;
b. transmitting by the sensors to a processing unit, leg positional information during at least motion of the at least one leg during a walking motion of the at least one leg;
c. the processing unit generating based on the leg positional information, data indicative of knee flexion angles and knee extension angles during at least the walking motion of the at least one leg;
d. the processing unit generating respective knee flexion and knee extension training signals based on the generated data;
e. receiving by at least one feedback unit, the generated training signals; and
f. providing feedback by the at least one feedback unit during at least the walking motion of the at least one leg based on the training signals, the feedback including at least a first component indicative of the subject's knee flexion angles during at least the walking motion of the at least one leg, and a second component indicative of the subject's knee extension angles during at least the walking motion of the at least one leg.

10. The method of claim 9 wherein the step of generating the training signals generates training signals indicative of knee flexion angles relative to corresponding predetermined knee flexion reference angles.

11. The method of claim 9 wherein the step of generating the training signals generates training signals indicative of knee extension angles relative to corresponding predetermined knee extension reference angles.

12. The method of claim 9 wherein the step of providing feedback comprises providing feedback by at least one of a display, projector, sound generator or haptic feedback.

13. The method of claim 9 further comprising enabling the subject to walk on a treadmill positioned in proximity to a device providing the feedback.

14. The method of claim 9, wherein the leg positional information transmitted by the sensors are indicative of three-dimensional segment accelerations and orientations.

15. The method of claim 9, wherein the step of releasably attaching sensors to at least one leg of the subject further comprises releasably attaching the sensors to respective one's thigh, shank and heel of the at least one leg of the subject.

16. The method of claim 15, wherein the step of releasably attaching the thigh sensor attaches such sensor at a location of about a midpoint of a line from the patella to the anterior superior iliac spine of the subject.

17. The method of claim 15, wherein the step of releasably attaching the shank sensor attaches such sensor at a location of about a prominent section of the gastrocnemius parallel to the thigh.

18. The method of claim 15, wherein the knee extension data comprises data concerning the subject's late swing, early, and mid-stance phases of a gait cycle.

19. The method of claim 15, wherein the knee flexion data comprises data concerning the subject's terminal stance, early and mid-swing phases of a gait cycle.

20. The method of claim 9, wherein the feedback providing step comprises providing feedback by at least a first output display indicative of a flexion error feedback needle.

21. The method of claim 20, wherein the feedback providing step comprises further providing feedback by a second output display indicative of an extension error feedback needle.
